(19) United States
(12) Patent Application Publication
(10) Pub. No.: US 2005/0177436 A1
(43) Pub. Date: Aug. 11, 2005

(54) Hierarchy for standard nomenclature

(75) Inventor: Anthony F. Menninger, Miami, FL (US)

Correspondence Address: FOLEY AND LARDNER;SUITE 500, 3000 K STREET NW, WASHINGTON, DC, 20007, US

(73) Assignee: Restaurant Services, Inc.

(21) Appl. No.: 10/959,306

(22) Filed: Oct. 7, 2004

Related U.S. Application Data
(60) Provisional application No. 60/509,897, filed on Oct. 10, 2003.

(51) Int. Cl. G06F 7/00; G06F 17/60

(52) U.S. Cl. 705/22; 705/28

(57) ABSTRACT

A data structure for the electronic reporting of point of sale data at a point of sale location to a supply chain management system utilizing a network is disclosed. The data structure includes a set of interconnected nodes for products comprising multiple interlocking hierarchies wherein at least one node has a plurality of parents from different hierarchies so that the at least one node is shared by a plurality of the hierarchies. A computer implemented method is also disclosed. The method is for creating a second hierarchy of nodes interconnected within an already existing hierarchy of nodes, the existing hierarchy of nodes including a first hierarchy of nodes different from the second hierarchy, for a supply chain management electronic reporting system utilizing a network.

### FIELD OF THE INVENTION

This invention relates to methods and structures for the electronic reporting of point of sale data at point of sale locations to a supply chain management system using hierarchies.

### BACKGROUND OF THE INVENTION

The organization of data using hierarchical indexing is known. It is also known to use such hierarchical indexing for organizing product data in an inventory system.

There is a need, however, in the context of point of sale (POS) data for point of sale locations within a supply chain, to allow for organization of the data according to different hierarchical organizations such that electronic reporting of the data is more easily facilitated.

### BRIEF DESCRIPTION OF THE DRAWINGS

The accompanying drawings, which are incorporated in and constitute a part of this specification, illustrate several embodiments of the invention and, together with the description, serve to explain the principles of the invention.

FIG. 1 illustrates an electronic reporting and feedback system according to an embodiment of the present invention.

FIG. 2 illustrates an example of a data structure incorporating interlocking hierarchies according to an embodiment of the present invention.

FIG. 3 is a flow chart illustrating the steps of creating a second hierarchy of nodes interconnected with an already existing hierarchy.

### DETAILED DESCRIPTION OF THE PREFERRED EMBODIMENTS

The present inventors have realized that there is a need to provide a data structure for reporting electronic point of sale data at a point of sale location to a supply chain management system, where the data structure has the flexibility to allow for multiple different hierarchies, where each of the hierarchies has different sets of nodes that have a parent-child relationship with at least some of the same child nodes. Thus, POS data may be reported and organized as desired by a user according to the particular hierarchy desired by the user. The multiple hierarchies are interlocked by having at least one of the child nodes shared by parent nodes of the different hierarchies.

The present inventors have also realized that efficiencies are realized by providing a method of creating a new hierarchy of nodes interconnected with a first hierarchy of an already existing hierarchy of nodes. By preserving the first hierarchy in creating the new hierarchy, the existing relationships between the nodes of the existing hierarchy are maintained. It is easier to maintain the interconnected hierarchy because one need only maintain the relationships between the nodes in a single place regardless of the number of hierarchies that make up the interconnected hierarchy. Also, a new hierarchy may be more easily created using an existing one, without the need to create a new hierarchy from the beginning.

The invention is described below with reference to drawings. These drawings illustrate certain details of specific embodiments that implement the systems and methods and programs of the present invention. However, describing the invention with drawings should not be construed as imposing on the invention any limitations that may be present in the drawings. The present invention contemplates methods, systems and program products on any computer readable media for accomplishing its operations. The embodiments of the present invention may be implemented using an existing computer processor, or by a special purpose computer processor incorporated for this or another purpose or by a hardwired system.

Embodiments within the scope of the present invention include program products comprising machine-readable media for carrying or having machine-executable instructions or data structures stored thereon. Such machine-readable media can be any available media which can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. When information is transferred or provided over a network or another communications connection (either hardwired, wireless, or a combination of hardwired or wireless) to a machine, the machine properly views the connection as a machine-readable medium. Thus, any such a connection is properly termed a machine-readable medium. Combinations of the above are also included within the scope of machine-readable media. Machine-executable instructions comprise, for example, instructions and data which cause a general purpose computer, special purpose computer, or special purpose processing machines to perform a certain function or group of functions.

Embodiments of the invention will be described in the general context of method steps which may be implemented in one embodiment by a program product including machine-executable instructions, such as program code, for example in the form of program modules executed by machines in networked environments. Generally, program modules include routines, programs, objects, components, data structures, etc. that perform particular tasks or implement particular abstract data types. Machine-executable instructions, associated data structures, and program modules represent examples of program code for executing steps of the methods disclosed herein. The particular sequence of such executable instructions or associated data structures represent examples of corresponding acts for implementing the functions described in such steps.

Embodiments of the present invention may be practiced in a networked environment using logical connections to one or more remote computers having processors. Logical connections may include a local area network (LAN) and a wide area network (WAN) that are presented here by way of example and not limitation. Such networking environments are commonplace in office-wide or enterprise-wide computer networks, intranets and the Internet and may use a wide variety of different communication protocols. Those skilled in the art will appreciate that such network computing environments will typically encompass many types of computer system configurations, including personal computers, hand-held devices, multi-processor systems, microprocessor-based or programmable consumer electronics, network PCs, minicomputers, mainframe computers, and the like. Embodiments of the invention may also be practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

An exemplary system for implementing the overall system or portions of the invention might include a general purpose computing device in the form of a conventional computer, including a processing unit, a system memory, and a system bus that couples various system components including the system memory to the processing unit. The system memory may include read only memory (ROM) and random access memory (RAM). The computer may also include a magnetic hard disk drive for reading from and writing to a magnetic hard disk, a magnetic disk drive for reading from or writing to a removable magnetic disk, and an optical disk drive for reading from or writing to a removable optical disk such as a CD-ROM or other optical media. The drives and their associated machine-readable media provide nonvolatile storage of machine-executable instructions, data structures, program modules and other data for the computer.

Referring again to the supply chain management system of the present invention, the organizational structure, technology applications and information systems that form portions of the Supply Chain are enablers that allow for effective management of the Supply Chain. The methodology of the present invention provides the means to efficiently capture, analyze and feed back timely Supply Chain data to the appropriate parties.

The claimed invention is applicable to many different industries, including but not limited to, pharmaceuticals, health and personal care products, computer and internet technology, automotive, home product supply, food and beverage, telecommunications, machinery, air conditioning and refrigeration, chemical, department store supply, office product supply, aircraft and airline related industries, education, consumer electronics, hotel, gasoline stations, convenience stores, restaurants, music and video, etc.

The present invention includes a supply chain management system involving at least one supply chain participant, and preferably multiple supply chain participants. Supply chain participants include a supply chain manager. The supply chain manager may be a supply chain participant, a department of, division of or consultant for a supply chain participant, or an independent entity unrelated to the other supply chain participants. The supply chain manager may be allowed to exercise management rights without taking title or possession of any goods passing through the supply chain.

Supply chain participants may also include brand owners, point of sale outlets, point of sale outlet owners, a cooperative or consortium of point of sale outlet owners, distributors, or suppliers. Suppliers may supply one or more of finished goods, partially finished goods or raw materials.

In general, the supply chain management system integrates various components, which components may include:
1. In-Retailer Systems
2. A Retailer/Distributor Electronic Interface
3. A Supplier/Distributor Electronic Interface
4. A Data Warehouse
5. Information Services
6. A Network such as a Web Architecture and Internet Access

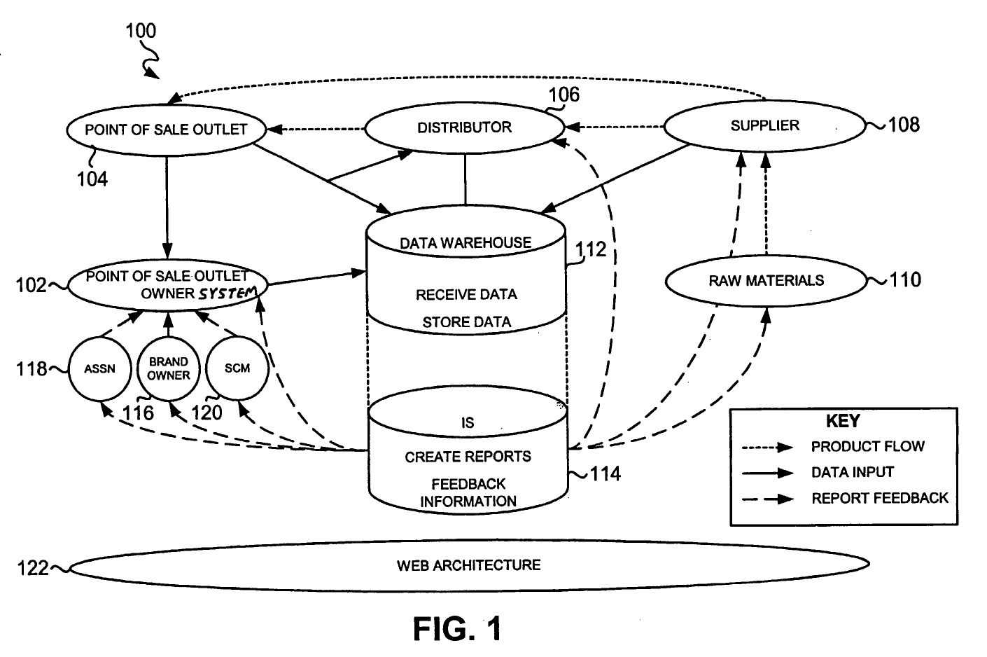

FIG. 1 illustrates an electronic reporting and feedback system 100 according to an embodiment of the present invention.

The In-Retailer Systems 102 support point of sale outlet owners with Point of Sale (POS) and Back of House (BOH) hardware and software solutions. This component enables electronic data collection of daily item sales for the information database. In the context of a restaurant, this collection of daily item sales might comprise menu sales. In the context of a supplier such as a factory, such sales might comprise inventory shipments out of the factory. Accordingly, for purposes of the present invention, the term “sales” is to be interpreted to include movement of product, either through retail sales of the product, or an inventory shipment out of a supplier.

The Retailer-Distributor Electronic Interface establishes an electronic purchasing system and thus “electronic commerce” between POS outlets 104 and distributors “direct” suppliers 106, 108. This includes electronic order entry over the network such as via the Web, order confirmation, product delivery/receiving, electronic invoicing, electronic wire payment transfers, data collection, and contract compliance and distributor performance measurement, which assists in managing distributor performance.

The Supplier-Distributor Electronic Interface facilitates the development of electronic commerce between system suppliers and distributors including electronic ordering and confirmations, electronic invoicing and payments and electronic supplier performance measuring and reporting. Electronic commerce between raw material suppliers 110 and suppliers is also provided.

A Data Warehouse 112 is a central collection point that electronically collects and warehouses timely, critical Supply Chain information for all Supply Chain participants. This includes distributor and supplier performance measures, representations of daily outlet item sales with translations to specified product requirements, and inventory levels, sales history and forecasts at various points in the Supply Chain, thereby providing a basis for collaborative planning and forecasting. The data stored in the Warehouse is then available for quick, secure access.

Information Services (IS) 114 analyzes, organizes and feeds back Supply Chain data to meet the information needs of Supply Chain users such as a brand owner 116, the Supply Chain Coordinator (SCC) 118, retail outlet management 120 and suppliers. This includes information on Supply Chain performance, collaborative planning and forecasting, promotion planning and inventory management. Services that benefit franchisees include electronic invoice auditing, distributor performance reporting, cost reporting and analysis, franchisee sales/cost comparables, and other reports. Information Services also determines a proper format in which to present the data so that it is in the most useful form for the end user. It also works with Supply Chain users to develop/evaluate analytical/operational tools.

A network 122 is provided to underlie this supply chain structure. In one embodiment the network may comprises a web architecture with Internet access (through proprietary service or an Internet Service Provider (ISP)) that allows these electronic communications to take place efficiently and effectively. Encompassed in this component is the building of initial web applications and security for the Supply Chain.

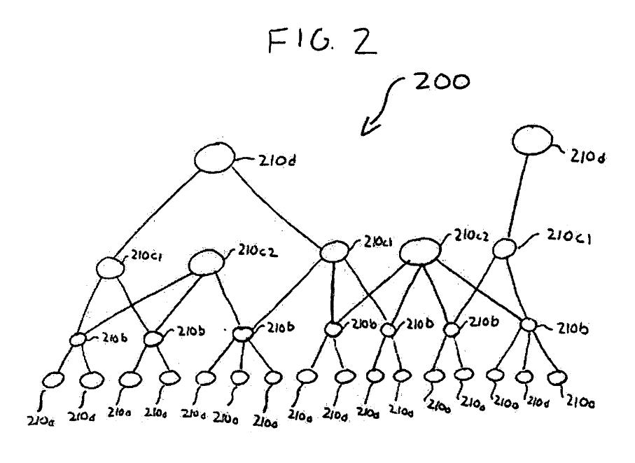

FIG. 2 illustrates an example of a data structure 200 for the electronic reporting of point of sale (POS) data at a point of sale location to a supply chain management system utilizing a network according to an embodiment of the invention. The data structure 200 includes a set of interconnected nodes 210a, 210b, 210c1, 210c2, 210d positioned at different levels of interlocking hierarchies. For simplicity, the data structure 200 in FIG. 2 illustrates only two different hierarchies. The present invention is not so limited, however, and may comprise more than two hierarchies.

In this application one hierarchy is considered different from another hierarchy if the nodes at a particular hierarchy level of the one hierarchy are of a different type than the nodes at the particular hierarchy level of the other hierarchy. For example, if the one hierarchy has nodes corresponding to distributors at a particular level, while the other hierarchy has nodes corresponding to geographic regions at the particular level, the one hierarchy is considered different than the other hierarchy. On the other hand, if each of the one hierarchy and the other hierarchy has nodes corresponding to distributors at the particular level, but the number of nodes at the particular level for the hierarchies is different, the one hierarchy is not considered different than the other hierarchy.

The interconnected nodes 210a, 210b, 210c1, 210c2, 210d are positioned at varying levels of their respective hierarchies. The nodes at a particular level have a parent-child relationship with one or more of the nodes at the next lower level. In FIG. 2, the existence of a parent-child relationship between one node at a particular level and another node at a next lower level is denoted by a line drawn from the one node to the another node. The parent-child relationship means that the one node is associated with the another node.

The leaf nodes 210a are at the first level (lowest level) of the interlocking hierarchies. In one embodiment, each of the leaf nodes 210a may represent products. For example, if the supply chain management system is for distribution of products to restaurants, the leaf nodes 210a may represent products for restaurants, such as food items, napkins, etc. Note that leaf nodes (not shown) could be at a lower level than the nodes 210a to represent components of the products. The nodes representing products would have a parent-child relationship with the nodes representing components of the products. When the products are food items, the components for products may be components of the food items. For example, if the food item is a cheeseburger, the components may include a hamburger pattie, slice of cheese, buns, etc. Also note that the hierarchies may include nodes (not shown) representing classifications of products. For example, the classification may be a classification of the product. For example, a cheeseburger may be classified as a food item. As another example, the classification may be the type of product, such as French food, Japanese food, etc., for example.

Nodes 210b are at the second level and are intermediate nodes. An intermediate node is a node between the leaf nodes and higher level nodes. Each of the nodes 210b has a parent-child relationship with one or more of the leaf nodes 210a. For example, if the supply chain management system is for distribution of products to point of sale locations, the intermediate nodes 210b may be point of sale location nodes representing point of sale locations. In this case, the leaf nodes 210a may represent products sold at a respective point of sale location, and there will be a parent-child relationship between a point of sale location node representing a point of sale location and leaf nodes representing products sold thereat. Taking the more specific example again where the supply chain management system is for distribution of products to restaurants, the intermediate nodes 210b may represent specific restaurants in the supply chain.

A first set of nodes 210c1 and a second set of nodes 210c2 are at the third level. The first set of nodes 210c1 and the second set of nodes 210c2 are of a different type, e.g., they correspond to different types of entities. The first set of nodes 210c1 is part of a first hierarchy, while the second set of nodes 210c2 is part of a second hierarchy, different from the first hierarchy. Both the nodes 210c1 and 210c2 are at the third level and have a parent-child relationship with one or more of the second level nodes 210b. Taking again the example where the supply chain management system is for distribution of products to point of sale locations, the first and second sets of nodes 210c1 and 210c2 may represent distributors and geographic regions, respectively. In this case for the first hierarchy with the first set of nodes 210c1, there will be a parent-child relationship between a node of the first set of nodes 210c1 representing a particular distributor and the next lower level intermediate nodes 210b representing point of sale locations for those point of sale locations that are serviced by the particular distributor.

For the second hierarchy with the second set of nodes 210c2, there will be a parent-child relationship between one of the second set of nodes 210c2 representing a particular geographic region and the next lower level intermediate nodes 210b representing point of sale locations for those point of sale locations that are within the particular geographic region. Each geographic region may be a state, for example, a plurality of states, or any other desired geographic region.

Nodes 210d are at a fourth level of the first hierarchy. Each of the nodes 210d has a parent-child relationship with one or more of the next lower level nodes of the first set of nodes 210c1. Taking again the example where the supply chain management system is for distribution of products to point of sale locations, the nodes 210d may represent suppliers. In this case, there will be a parent-child relationship between a node 210d representing a particular supplier and the nodes of the next lower level first set of nodes 210c1 representing distributors for those distributors that are serviced by the particular supplier.

The first hierarchy of the data structure 200 illustrated in FIG. 2 includes the nodes 210a, 210b, 210c1 and 210d, while the second hierarchy includes the nodes 210a, 210b and 210c2. Thus the data structure 200 comprises a set of interconnected nodes comprising multiple interlocking hierarchies wherein at least one node has a plurality of parents from different hierarchies so that the at least one node is shared by a plurality of the hierarchies. In the data structure specifically illustrated in FIG. 2, the interlocking hierarchies are the first hierarchy and the second hierarchy. In this case, at least some of the intermediate nodes 210b have a plurality of parents, because at least some of the intermediate nodes 210b may have one parent from the first set of nodes 210c1 of the first hierarchy and another parent from the second set of nodes 210c2 of the second hierarchy.

While FIG. 2 illustrates the second hierarchy as not including the fourth level nodes 210d, alternatively, the second hierarchy may also include one or more of the fourth level nodes 210d. For example, if the second set of nodes 210c2 represents geographical regions and the nodes 210d represent suppliers as described above, the second hierarchy may indicate a parent-child relationship between a node 210d representing a particular supplier and a node of the second set of nodes 210c2 representing the geographic region containing the distributors serviced by the particular supplier.

While FIG. 2 illustrates an example with interconnected hierarchies, where the total number of levels for the interconnected hierarchies is 4, the present invention is not so limited. In general the number of levels for each hierarchy may be more or less than 4, and the number may be as low as two, for example. Also, in general, the number of levels of the different hierarchies may be the same or different.

In one embodiment of the invention, each node of the data structure includes the following fields:

Level: height of the node (e.g., leaf nodes are at level one, intermediate nodes at level two and possibly higher, etc.)

Node_id: unique node identifier

Child_count: the number of next lower level nodes for which the node has a parent-child relationship

Parent: identifies (via a pointer) the node's parent

Leaf element: for leaf nodes, the product, product component, and/or characteristics.

The data structure 200 may be implemented by being stored in a database, as is known in the art.

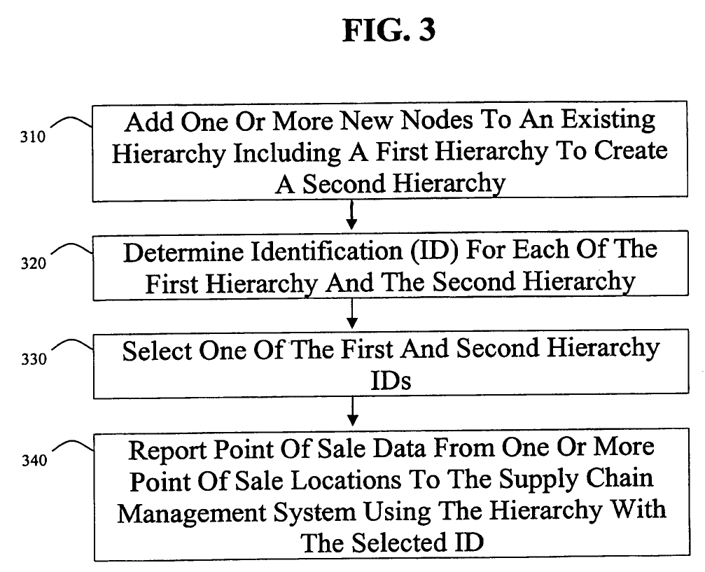

FIG. 3 is a flow chart illustrating a computer implemented method for creating a second hierarchy of nodes interconnected within an already existing hierarchy of nodes, the existing hierarchy of nodes including a first hierarchy of nodes, for a supply chain management electronic reporting system utilizing a network. The existing hierarchy includes at least the first hierarchy of nodes, but may include additional hierarchies, such as a third hierarchy, of nodes different from each of the first and second hierarchies of nodes. The method may be implemented by a user utilizing the network, for example. The user, for example, may implement the method via a user work station including a display and an input device, such as a keyboard and/or a mouse.

The second hierarchy and first hierarchy in the method illustrated in FIG. 3 may be the second hierarchy and first hierarchy, respectively, illustrated in FIG. 2, for example. If the existing hierarchy includes a third hierarchy as well as the first hierarchy, where the first hierarchy includes nodes representing distributors, the second hierarchy includes nodes representing geographic regions, the third hierarchy may include nodes representing point of sale location product sales volume size, for example. The product volume sales sizes may be, for example, small (below a certain value), large (above another value), and medium (between the small and large sizes).

Referring to step 310 in FIG. 3, one or more new nodes are added to the already existing hierarchy to create the second hierarchy. Take again, for example, the case where a second hierarchy to be interlocked with the first hierarchy, such as illustrated in Example 2, is to be created, and where the supply chain management system is for distribution of products to point of sale locations. In this case, the nodes at a level of interest in the first hierarchy may represent distributors while the one or more new nodes being added to create the new hierarchy, for example, may represent respective geographical regions.

In one embodiment, a user may add the one or more new nodes by utilizing the user work station with its attendant display and input device. For example, the user may be prompted at the user work station as to whether the user desires to create a new hierarchy, and further prompted to add the one or more new nodes to create the second hierarchy. The user then adds the one or more new nodes, such as nodes representing one or more geographical regions.

In creating the second hierarchy, the user need not add nodes representing all of the geographical regions which include a point of sale location corresponding to an existing node of the existing hierarchy. Thus, the method provides flexibility in creating the second hierarchy as desired by the user. For example, it may be desired to have information for only some geographical regions containing point of sales locations. Of course, if desired, a user may add nodes representing each of the geographical regions that include a point of sale location corresponding to an existing node of the existing hierarchy.

In creating the second hierarchy by adding at least one node, the at least one node may also branch back into the existing hierarchy as a child in a parent-child relationship. Taking again, for example, the case where a second hierarchy, for interlocking first and second hierarchies such as those illustrated in FIG. 2, is to be created, the at least one added node, added as a node at the third level, may branch back to a node at the fourth level. For example, the added nodes may branch back to nodes representing suppliers.

Referring to step 320, an identification (ID) is determined for each of the first hierarchy and the second hierarchy. In one embodiment, a user may perform this step at the user work station. For example, after, concurrently with, or before the second hierarchy is created, the user may be prompted to provide an ID for each of the first hierarchy and the second hierarchy, and may do so using an input device at the user work station. It is also possible that prior to the second hierarchy being created, the first hierarchy may already have an ID. Also, the second hierarchy ID may be determined before, currently with, or after creating the second hierarchy. The ID may be an ID chosen by the user, such as a name, for example, or it may be an ID determined by the system, or at random.

Referring to step 330, one of the hierarchy IDs is selected. In one embodiment a user may select the ID for one of the second hierarchy and first hierarchy at a user work station using an input device. The user may be prompted by a menu on a display listing hierarchy IDs including at least IDs for the first hierarchy and the second hierarchy. The menu may be a pull-down menu, for example. The user selects the ID and the selection is received within the network to determine the ID for the hierarchy.

In general, steps 310 and 320, which respectively create the second hierarchy and determine IDs for both the second hierarchy and the first hierarchy, may be performed by a user managing the hierarchies, while the step 330 may be performed by a user who desires particular information obtainable using one of the hierarchies. All of the steps may also be performed by the same user.

As an alternative to the user directly selecting a desired ID, a hierarchy ID may be automatically selected based on a criteria. In one embodiment, the criteria may be a point of sale location. In this case, a user may select a desired point of sale location. A hierarchy ID is then automatically selected based on the point of sale location. For example, if the second hierarchy does not include the point of sale location, the first hierarchy ID may be automatically selected.

Referring to step 340, point of sale data is reported from one or more point of sale locations to the supply chain management system using the hierarchy with the selected ID. The point of sale data for the products or product components sold at the point of sale location may be reported according to the relationships of the selected hierarchy and a desired format. For example, if the second hierarchy is selected where the second hierarchy includes nodes representing geographic regions, a report may be generated listing the point of sale data for each product, product component, or characteristic, or a combination as desired, according to geographic region. As another example, if the first hierarchy is selected where the first hierarchy includes nodes representing distributors, a report may be generating listing the point of sale data for each product, product component, or characteristic according to distributors. Depending upon the hierarchy ID selected, point of sale data is reported using either the second hierarchy or the first hierarchy.

As discussed above, in general the number of different hierarchies may be greater than two. In this case, an ID may be determined for each of the hierarchies, and subsequently, a desired hierarchy ID may be selected for reporting point of sale data.

It should be noted that although the flow charts provided herein show a specific order of method steps, it is understood that the order of these steps may differ from what is depicted. Also two or more steps may be performed concurrently or with partial concurrence. Such variation will depend on the software and hardware systems chosen and on designer choice. It is understood that all such variations are within the scope of the invention. Likewise, software and web implementations of the present invention could be accomplished with standard programming techniques with rule based logic and other logic to accomplish the various database searching steps, correlation steps, comparison steps and decision steps.

The foregoing description of embodiments of the invention has been presented for purposes of illustration and description. It is not intended to be exhaustive or to limit the invention to the precise form disclosed, and modifications and variations are possible in light of the above teachings or may be acquired from practice of the invention. The embodiments were chosen and described in order to explain the principals of the invention and its practical application to enable one skilled in the art to utilize the invention in various embodiments and with various modifications as are suited to the particular use contemplated.

1. A data structure for the electronic reporting of point of sale data at a point of sale location to a supply chain management system utilizing a network, comprising:
a set of interconnected nodes for products comprising multiple interlocking hierarchies wherein at least one node has a plurality of parents from different hierarchies so that the at least one node is shared by a plurality of the hierarchies.

2. The data structure as defined in claim 1, wherein one of the multiple hierarchies was formed by adding to an existing hierarchy at least one node as one of the parents of the plurality of parents, with that added node branching back into the existing hierarchy as a child in a parent-child relationship.

3. The data structure as defined in claim 1, wherein the different hierarchies include a first hierarchy with a first set of nodes representing distributors, and a second hierarchy with a second set of nodes representing geographic regions, the first hierarchy being different from the second hierarchy.

4. The data structure as defined in claim 3, wherein the different hierarchies include a plurality of point of sale location nodes representing point of sale locations, wherein the first set and second set of nodes have parent-child relationships with respective ones of the point of sale location nodes.

5. The data structure as defined in claim 4, wherein the different hierarchies include a plurality of leaf nodes, each leaf node representing products, wherein the point of sale location nodes have a parent-child relationship with respective ones of the leaf nodes.

6. The data structure as defined in claim 5, wherein the first hierarchy further includes nodes representing suppliers having a parent-child relationship with respective ones of the first set of nodes representing distributors.

7. The data structure as defined in claim 1, wherein the number of hierarchies in the multiple interlocking hierarchies is two.

8. The data structure as defined in claim 1, wherein the number of hierarchies in the multiple interlocking hierarchies is greater than two.

9. The data structure as defined in claim 1, wherein the set of interconnected nodes further comprises nodes representing product components of the products or of classifications of the products.

10. A computer implemented method for creating a second hierarchy of nodes interconnected within an already existing hierarchy of nodes, the existing hierarchy of nodes including a first hierarchy of nodes different from the second hierarchy, for a supply chain management electronic reporting system utilizing a network, comprising:
adding one or more new nodes to the already existing hierarchy to create the second hierarchy interlocked with the first hierarchy;
determining an ID for each of the first hierarchy and the second hierarchy; and
selecting one of the hierarchy IDs and reporting point of sale data from a point of sale location to the supply chain management system using the hierarchy with the selected ID.

11. The method as defined in claim 10, wherein at least one node is shared between the first and second hierarchies and branches back to respective nodes in the first and second hierarchies not shared by the first and second hierarchies.

12. The method as defined in claim 10, wherein the selecting step comprises displaying a menu listing at least the first and second hierarchies to a user; and
receiving a selection from the user.

13. The method as defined in claim 10, wherein the selecting step comprises automatically selecting one of the hierarchy IDs based on a criteria.

14. The method as defined in claim 13, wherein the criteria is a location of the point of sale.

15. The method as defined in claim 10, wherein the first hierarchy includes a first set of nodes representing distributors, and wherein the added nodes comprise a second set of nodes representing geographic regions.

16. The method as defined in claim 15, wherein both the first and second hierarchies include a plurality of point of sale location nodes representing point of sale locations, wherein the first set and second set of nodes have parent-child relationships with respective ones of the point of sale location nodes.

17. The method as defined in claim 16, wherein both the first and second hierarchies include a plurality of leaf nodes, each leaf node representing products, wherein the point of sale location nodes have a parent-child relationship with respective ones of the leaf nodes.

18. The method as defined in claim 17, wherein both the first and second hierarchies further comprise nodes representing product components of the products or classifications of the products.

19. The method as defined in claim 17, wherein the first hierarchy further includes nodes representing suppliers having a parent-child relationship with respective ones of the first set of nodes representing distributors.

20. The method as defined in claim 10, wherein the already existing hierarchy of nodes includes a third hierarchy of nodes different from the first and second hierarchies.

21. The method as defined in claim 10, wherein the ID for each of the first hierarchy and the second hierarchy is determined after creating the second hierarchy.

22. The method as defined in claim 10, wherein the ID for the first hierarchy is determined before creating the second hierarchy, and the ID for the second hierarchy is determined after creating the second hierarchy.
